SEQUENCE LISTING (8 sequences)

SEQ ID NO: 1 - RNA, 96 nt, Dengue virus
  source 1..96: mol_type = mRNA
agttgttagtctacgtggaccgacaaagacagattctttgagggagctaagctcaacgtagttctaacagttttttaattagagagcagatctctg

SEQ ID NO: 2 - RNA, 469 nt, Ebola virus
  source 1..469: mol_type = mRNA
cggacacacaaaaagaaagaagaatttttaggatcttttgtgtgcgaataactatgaggaagattaataattttcctctcattgaaatttatatcggaatttaaattgaaattgttactgtaatcacacctggtttgtttcagagccacatcacaaagatagagaacaacctaggtctccgaagggagcaagggcatcagtgtgctcagttgaaaatcccttgtcaacacctaggtcttatcacatcacaagttccacctcagactctgcagggtgatccaacaaccttaatagaaacattattgttaaaggacagcattagttcacagtcaaacaagcaagattgagaattaaccttggttttgaacttgaacacttaggggattgaagattcaacaaccctaaagcttggggtaaaacattggaaatagttaaaagacaaattgctcggaatcacaaaattccgagt

SEQ ID NO: 3 - RNA, 292 nt, HCov 2229E virus
  source 1..292: mol_type = mRNA
acttaagtaccttatctatctacagatagaaaagttgctttttagactttgtgtctacttttctcaactaaacgaaatttttgctatggccggcatctttgatgctggagtcgtagtgtaattgaaatttcatttgggttgcaacagtttggaagcaagtgctgtgtgtcctagtctaagggtttcgtgttccgtcacgagattccattctacaaacgccttactcgaggttccgtctcgtgtttgtgtggaagcaaagttctgtctttgtggaaaccagtaactgttccta

SEQ ID NO: 4 - RNA, 335 nt, HI virus
  source 1..335: mol_type = mRNA
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagag

SEQ ID NO: 5 - RNA, 278 nt, MERS Co virus
  source 1..278: mol_type = mRNA
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatc

SEQ ID NO: 6 - RNA, 264 nt, SARS Co virus
  source 1..264: mol_type = mRNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaag

SEQ ID NO: 7 - RNA, 265 nt, SARS Co virus 2
  source 1..265: mol_type = mRNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 8 - RNA, 106 nt, Zika virus
  source 1..106: mol_type = mRNA
agttgttgatctgtgtgagtcagactgcgacagttcgagtctgaagcgagagctaacaacagtatcaacaggtttaatttggatttggaaacgagagtttctggtc

(19) United States
(12) Patent Application Publication
(10) Pub. No.: US 2026/0272874 A1
(43) Pub. Date: Sep. 17, 2026

(54) COMPOUNDS FOR THE TREATMENT AND PREVENTION OF VIRAL DISEASES

(71) Applicant: SJP Biotech GmbH, Meilen (CH)

(72) Inventor: Krishnaraj Rajalingam, Nieder-Olm (DE)

(21) Appl. No.: 19/168,921

(22) PCT Filed: Mar. 27, 2024
(86) PCT No.: PCT/EP2024/058186

§ 371 Date: Sep. 25, 2025

(30) Foreign Application Priority Data
Mar. 30, 2023 (EP) 23165567.1

(51) Int. Cl. A61K 31/343

(52) U.S. Cl. CPC A61K 31/343

(57) ABSTRACT

The present invention relates to new therapeutic uses of 1H-Cyclopenta[b]benzofuran derivatives, specifically new uses for the treatment or prevention of viral infections or a disease associated with viral infections and/or for use as inhibitor of viruses. The invention further relates to a method of treatment or prevention of viral infections or diseases associated with viral infections. Moreover, the invention relates to a pharmaceutical composition for use in the treatment or prevention of viral infections or diseases associated with viral infections.

The present invention relates to new therapeutic uses of 1H-Cyclopenta[b]benzofuran derivatives, specifically new uses for the treatment or prevention of viral infections or a disease associated with viral infections and/or for use as inhibitor of viruses. The invention further relates to a method of treatment or prevention of viral infections or diseases associated with viral infections. Moreover, the invention relates to a pharmaceutical composition for use in the treatment or prevention of viral infections or diseases associated with viral infections.

### BACKGROUND OF THE INVENTION

Viral infections pose a major challenge for public health systems throughout the world. Despite rapid scientific progress in the field of virology, the lack of effective therapeutic options and vaccines for most viral infectious diseases continues to take its toll. Emerging viral diseases pose a major threat primarily because of their rapid transmission rates and the inherent difficulties associated with developing effective vaccines and treatment options against such novel pathogens in a short span of time. Hence there is a need to develop broad-spectrum antiviral compounds that are effective against multiple viruses. The majority of currently available antivirals target one or more components directly associated with the virus replication cycle. Hence their activity tends to be limited to closely related viruses, often within the same virus family. Selected nucleoside analogues including cidofovir, favipiravir and ribavirin have shown to be effective against a broad range of viruses but pose a major risk of developing antiviral resistance mainly among RNA viruses.

Viruses are infectious organic structures that spread as virions outside cells (extracellular) by transmission, but as viruses can only replicate within a suitable host cell (intracellular). They do not themselves consist of one or more cells. All viruses contain the program for their replication and spread (some viruses also contain other auxiliary components), but have neither independent replication nor their own metabolism and are therefore dependent on the metabolism of a host cell. The viruses attach to surface molecules of the host cells and introduce their genetic material into them. This penetrates into the cell nucleus and alters the cell's own DNA. The virus body (genome and proteins) replicates, sometimes on a massive scale, in the infected cell through the existing cell organelles.

A virus particle outside cells is called a virion. Virions are particles that contain nucleic acids—either deoxyribonucleic acids (DNA) or ribonucleic acids (RNA)—and usually have an enclosing protein capsule (capsid). However, a capsule is absent in the influenza virus, for example, which has a ribonucleoprotein instead. Some virions additionally possess an envelope by a bio membrane whose lipid bilayer is interspersed with viral membrane proteins. This is referred to as the viral envelope. Virions that temporarily have a viral envelope in addition to the capsid until the replication phase begins are referred to as enveloped, and viruses without such an envelope are referred to as non-enveloped.

R. Madhugiri et al., Advances in Virus Research, Vol. 96, 2016, 127, summarizes the coronavirus RNA synthesis and discuss the structural and functional features of known cis-acting RNA elements located in the 5′- and 3′-terminal untranslated region (UTR).

Previous studies have shown that Silvesterol (CAS 697235-38-4), a flavagline derivative acts as a potent and selective inhibitor of the RNA helicase enzyme eIF4A and has broad-spectrum antiviral activity against diseases such as Ebola and coronaviruses. Silvesterol was difficult to synthesize due to the cyclo-penta-benzofuran ring and thus several modified synthetic flavaglines have been designed and successfully employed at least for in vitro preclinical studies. A couple of studies have already shown that Silvesterol has broad spectrum antiviral activity against corona viruses, SARS and MERS virus (C. Müller et al., Antiviral research 150, 2018, 123).

Wintachai et al., Microbiol Immunol., 2015, 59, 129-141 relates to the assessment of flavaglines, in particular FL3 and FL23 as chikungunya virus inhibitor.

Similar studies have been conducted with further eIF4A inhibitors. R. Cencic et al., J. Virology, 2011, 6381 describes that the compound 4E2RCat (432499-63-3), an inhibitor of eIF4E-eIF4G interaction, is capable of blocking coronavirus replication.

E. Gordon et al., bioRxiv Mar. 22, 2020, relates to the searching for knowledge of the molecular details of SARS-CoV-2 infection. Therefore, viral proteins in human cells have been cloned, tagged and expressed. Further, 66 druggable human proteins or host factors targeted by 69 existing FDA-approved drugs, drugs in clinical trials and/or preclinical compounds have been identified inter alia Zotatifin (eFT226). The compounds are evaluated efficacy in live SARS-CoV-2 infection assays.

EFFECTOR Therapeutics, Apr. 30, 2020, have reported that Zotatifin, an oncology product, shows antiviral activity against SARS-CoV-2.

However, there is still a great demand for further compounds that exhibit a broad-spectrum of antiviral properties.

It is therefore an object of the present invention to provide pharmaceutically active compounds that have the capability to inhibit virus activity.

### SUMMARY OF THE INVENTION

The invention relates to a compound of formula (I)

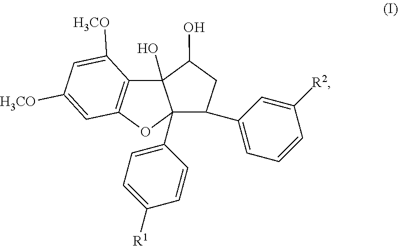

prodrugs or isotopically enriched forms thereof or a pharmaceutically acceptable salt or solvate thereof, wherein
R¹ is selected from CN and hydrogen,
R² is selected from hydrogen and halogen,
for use in the treatment or prevention of viral infections or diseases associated with viral infections.

In particular, the invention relates to a compound of formula (I)

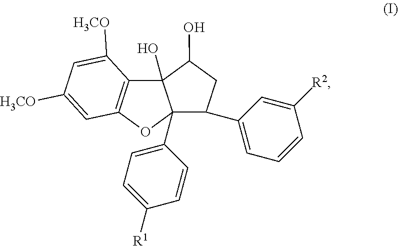

prodrugs or isotopically enriched forms thereof or a pharmaceutically acceptable salt or solvate thereof, wherein
R¹ is selected from CN and hydrogen,
R² is selected from hydrogen and halogen,
for use in treatment or prevention of viral infections or diseases associated with viral infections infection and/or for use as inhibitor of viruses, wherein the infection or disease is selected from viral infection caused by positive single-stranded RNA viruses ((+) ssRNA) and a negative single-stranded RNA viruses ((−) ssRNA).

The invention further relates to a compound of formula (I), as defined above and below, for use as inhibitor of viruses.

The invention further relates to compounds of formula (I.a) or (I.b) or an enantiomeric mixture comprising the compounds of formula (I.a) and (I.b) or prodrugs or isotopically enriched forms thereof or the pharmaceutically acceptable salts or solvates thereof, for use in the treatment or prevention of viral infections or diseases associated with viral infections, in particular, wherein the infection or disease is selected from viral infection caused by positive single-stranded RNA viruses ((+) ssRNA) and a negative single-stranded RNA viruses ((−) ssRNA)

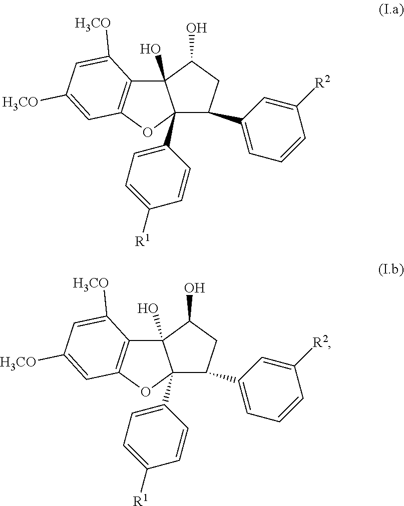

wherein R¹ and R² have the same meanings as defined above and below.

The invention further relates to compounds of formula (I.a) or (I.b) or an enantiomeric mixture comprising the compounds of formula (I.a) and (I.b) or prodrugs or isotopically enriched forms thereof or the pharmaceutically acceptable salts or solvates thereof, for use as inhibitor of viruses

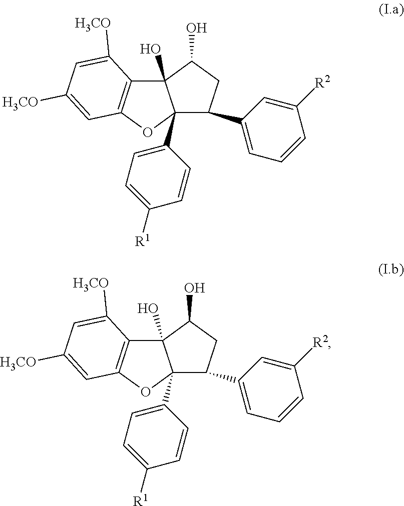

wherein R¹ and R² have the same meanings as defined above and below.

The invention further relates to a method of treatment or prevention of viral infections or diseases associated with viral infections, as defined above and below comprising administering a therapeutically effective amount of at least one compound selected from compounds of formulae (I), (I.a), (I.b), an enantiomeric mixture comprising the compounds of formula (I.a) and (I.b) or prodrugs or isotopically enriched forms thereof or a pharmaceutically acceptable salt or solvate thereof as defined herein.

The invention further relates to a pharmaceutical composition comprising a pharmaceutical effective amount of at least one compound selected from compounds of formulae (I), (I.a), (I.b), an enantiomeric mixture comprising the compounds of formula (I.a) and (I.b) or prodrugs or isotopically enriched forms thereof or a pharmaceutically acceptable salt or solvate thereof, as defined herein, and one or more pharmaceutical acceptable or carrier for use in the treatment or prevention of viral infections or diseases associated with viral infections.

The invention further relates to a pharmaceutical composition comprising a pharmaceutical effective amount of at least one compound selected from compounds of formulae (I), (I.a), (I.b), an enantiomeric mixture comprising the compounds of formula (I.a) and (I.b) or prodrugs or isotopically enriched forms thereof or a pharmaceutically acceptable salt or solvate thereof, as defined herein, and one or more pharmaceutical acceptable or carrier for use as inhibitor of viruses.

### DESCRIPTION OF THE INVENTION

The invention has the following advantages:
The compounds according to the invention exhibit advantageous virus inhibition. (+) ssRNA viruses like coronaviruses and picocorona viruses are dependent on the host 5′ Cap dependent and Cap independent strategies for viral mRNA translation initiation.
Some viruses exploit the plasma membrane associated prohibitins for their entry into host cells. The current invention describes class of molecules that target both PHBs in the plasma membrane and eIF4A complex and thereby preventing viral entry and replication.

### Compounds of Formula (I)

Unless specifically stated otherwise herein, references made in the singular may also include the plural. For example, “a” and “an” may refer to either one, or one or more.

The expression “halogen” denotes in each case fluorine, chlorine, bromine or iodine, preferably fluorine.

The term “solvate” refers to a complex of a compound according to the invention that is coordinated to a solvent molecule to form a specific ratio, in particular solvate is “hydrate”, which means a complex formed by the coordination of a compound of the invention with water.

The compounds of formulae (I), (I.a), (I.b), (A), (B), an enantiomeric mixture thereof, may form salts which are also within the scope of this invention. The term “salt(s)” as employed herein, denotes acidic and/or basic salts formed with inorganic and/or organic acids and bases. Pharmaceutically acceptable (i.e. non-toxic, physiologically acceptable) salts are preferred, although other salts are also useful, e.g., in isolation or purification steps which may be employed during preparation. Salts of the compounds of formulae (I), (I.a), (I.b), (A), (B), or an enantiomeric mixture thereof may be formed, for example, by reacting a compound of formulae (I), (I.b), (A), (B), or an enantiomeric mixture thereof with at least one acid or base. The acid or base is added in an amount suitable for partial or complete neutralization, e.g. an equivalent amount.

The phrase “pharmaceutically acceptable salt(s)” as used herein, unless otherwise indicated, includes salts containing pharmacologically acceptable anions or cations, such as chloride, bromide, iodide, nitrate, sulfate, bisulfate, phosphate, hydrogen phosphate, dihydrogen phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, sulfate, benzenesulfonate, p-toluenesulfonate and palmoate [i.e. 4,4′-methylene-bis-(3-hydroxy-2-naphthoate)] salts.

Any compound according to the invention can be prepared and used in prodrug form. A suitable prodrug has chemically or metabolically cleavable group(s) and becomes, by solvolysis or under physiological conditions, a compound that is pharmaceutically active in vivo. A prodrug can be formed in a conventional manner by reaction of a functional group of the compound (such as an amino, hydroxy or carboxy group). Prodrugs often offer advantages of better metabolism, potency, solubility, tissue compatibility, or delayed release in mammals.

The term “prodrug” refers to a metabolic precursor of a compound of the invention that is pharmaceutically acceptable. A prodrug may be inactive when administered to a subject in need thereof but is converted in vivo to an active compound of the invention. Prodrugs are typically rapidly transformed in vivo to yield the parent compound of the invention, for example, by hydrolysis in blood.

In other words, in the terms of the present invention, “prodrugs” are compounds which are metabolized in vivo to give the compounds of the invention of formula I. Typical examples for prodrugs are for example described in C. G. Wermeth (editor): The Practice of Medicinal Chemistry, Academic Press, San Diego, 1996, pages 671-715 as well as J. Rautio et al., NATURE REVIEWS, Drug Discovery, Vol. 17, 2018, p. 559. Examples are phosphates, carbonates, carbamates, aminoacids, esters (carboxylic acid esters), ethers, amides, peptides, ureas and the like. In the present case, suitable prodrugs can be compounds of formula I wherein the hydroxy group is bonded to any group that, when the prodrug of the compound of the invention is administered to a mammalian subject, cleaves to form a free hydroxy group.

The terms “virus infections” and “viral infections” relates to the entry of viruses into an organism as well as their colonization and multiplication. The resulting disease is the viral infection.

The term “diseases associated with viral infections” relates to diseases resulting from a viral infection. Associated disease is preferably selected from lung inflammation, respiratory distress, pulmonary fibrosis, pneumonia, cytokine storm, acute liver injury, septic shock, acute kidney injury, pancreatic injury, peripheral nervous systems complications (such as an impaired ability to taste, to smell, and vision impairment), muscle pain, inflammation of cardiac muscle, blood clots in veins, decreased blood flow in coronary arteries, cardiogenic shock, heart failure, impaired consciousness, brain inflammation, irritation and swelling of brain and blood vessels, acute cerebrovascular complications (such as stroke, seizures and slurred speech), arrhythmia, myocarditis, thrombotic events rhabdomyolysis, neurocognitive deficits, cancer, and sensory and motor deficits.

In the context of the present invention, a chemical structure that does not explicitly show a specific stereochemical orientation usually means all possible stereoisomers and mixtures thereof, unless indicated otherwise, for example,

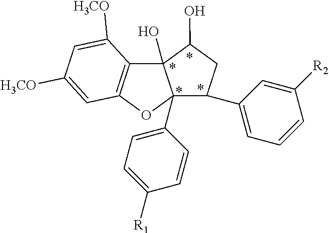

in which * designates the chiral centers.

“Chiral compounds” in the sense of the invention are compounds that contain no improper axis of rotation (Sn). In the context of the present invention, they are in particular compounds with at least four chirality centers and without Sn-symmetry.

“Stereoisomers” in the context of the invention are compounds of identical constitution but different atomic arrangement in the three-dimensional space.

“Enantiomers” are stereoisomers which behave like mirror to images to one another, e.g. compounds of formulae (I.a) and (I.b) are enantiomers. The “enantiomeric excess” (ee) achieved during asymmetric synthesis is given here by the following formula: ee [%]=(R−S)/(R+S)×100.

R and S are the descriptors of the CIP system for the configuration of a stereocenter to describe the absolute configuration at an asymmetric (carbon) atom. The enantiomerically pure compound (ee=100%) is also referred to as a “homochiral compound”.

“Diastereomers” are stereoisomers which are not enantiomeric to one another.

The compound of the invention can exist in various isomeric forms, as well as in one or more tautomeric forms, including both single tautomers and mixtures of tautomers. The term “isomer” is intended to encompass all isomeric forms of a compound of this invention, including tautomeric forms of the compound.

Some compounds described here can have asymmetric centers and therefore exist in different enantiomeric and diastereomeric forms. A compound of the invention can be in the form of an optical isomer or a diastereomer. Accordingly, the invention encompasses compounds of the invention and their uses as described herein in the form of their optical isomers, diastereoisomers and mixtures thereof, including a racemic mixture. Optical isomers of the compounds of the invention can be obtained by known techniques such as asymmetric synthesis, chiral chromatography, or via chemical separation of stereoisomers through the employment of optically active resolving agents.

Unless otherwise indicated “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

Compounds of the invention or their pharmaceutically acceptable salts or solvates may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography.

Relative configuration in stereochemistry (relative stereochemistry) is the arrangement of atoms or groups of atoms that is described relative to other atoms or groups of atoms in the molecule. In other words, this term describes the position of atoms or groups of atoms in space in relation to other atoms or groups of atoms that are located elsewhere in the molecule.

Absolute configuration in stereochemistry (absolute stereochemistry) is the arrangement of atoms or group of atoms that is described independently of any other atom or group of atoms in the molecule. This type of configuration is defined for chiral molecular entities and their stereochemical descriptions (e.g. R or S).

Syn means that with regard to the orientation of the substituents on the 5-membered ring they are bound to (4 asymmetric carbon atoms) all substituents point in the same direction relative to the plane of the 5-membered ring.

Racemic mixture or racemate is defined as a mixture of compounds consisting of two molecules structured like image and mirror image (=enantiomers) and which are present in an equimolar mixture, i.e. in the ratio 1:1 (50:50).

Further, the compounds of formula (I)

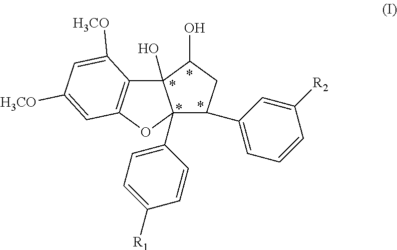

in which * designates the asymmetry centers, represent the isomers of formulae (I.a), (I.b), (I.c), (I.d), (I.e), (I.f), (I.g), (I.h), (I.i), (I.j), (I.k), (I.l), (I.m), (I.n), (I.o) and (I.p):

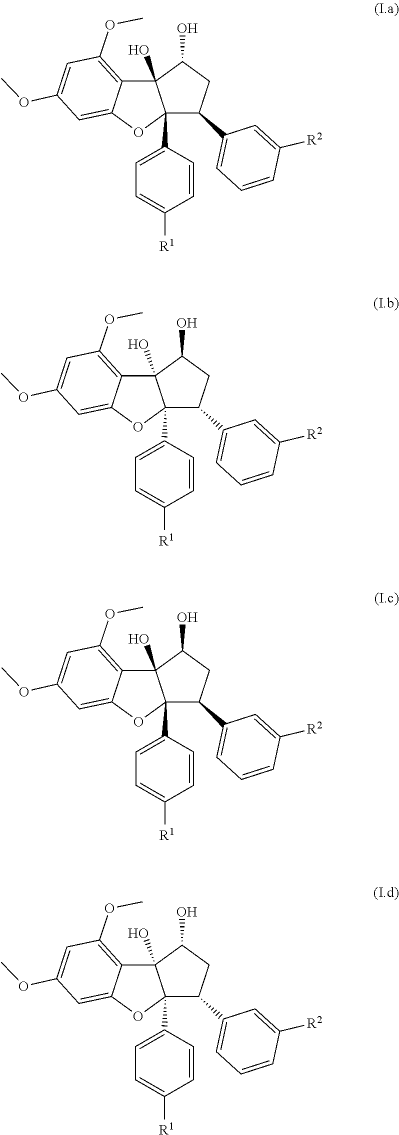

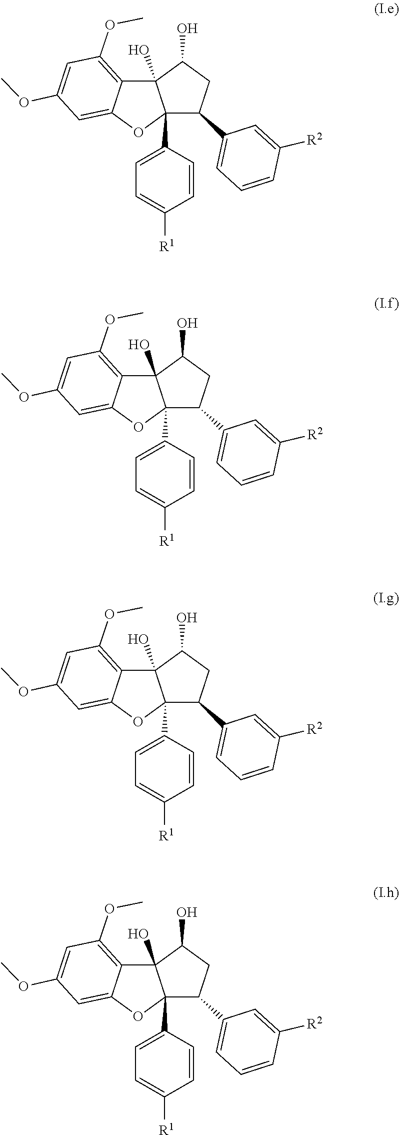

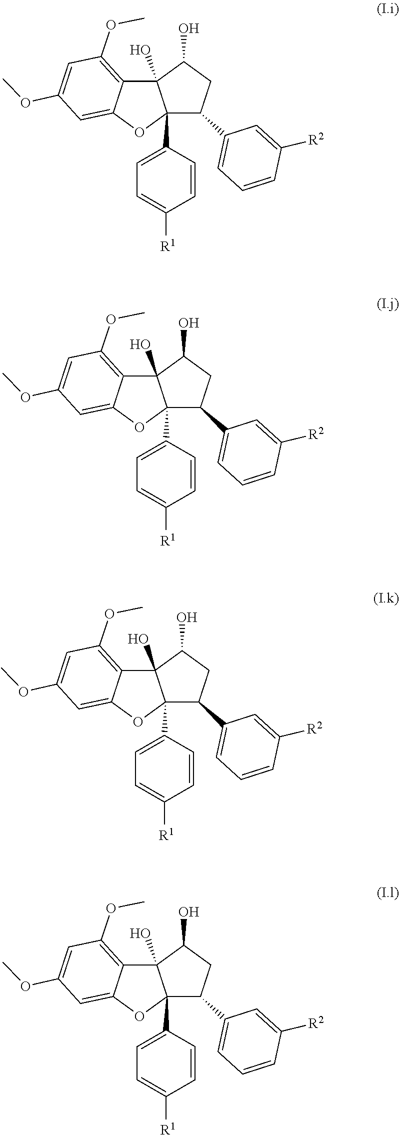

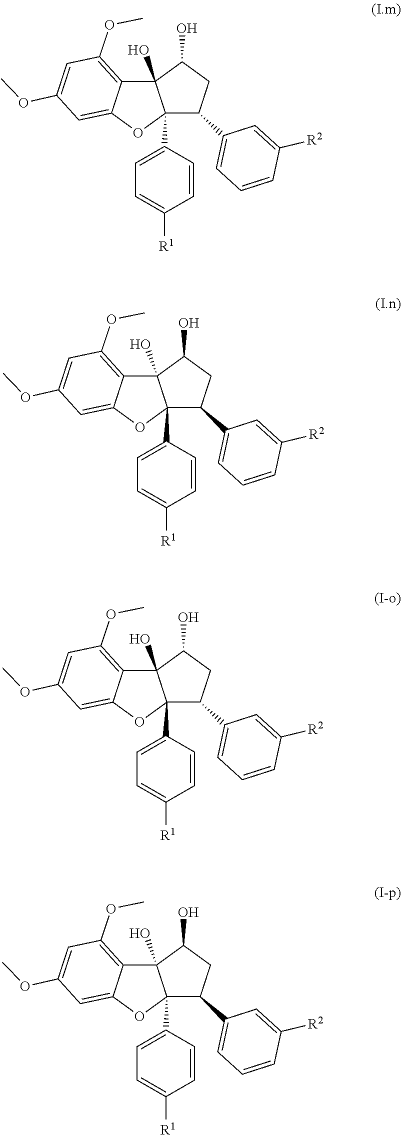

wherein, R¹ and R² have one of the meanings as defined above or below.

The compounds of formulae (I.a) to (I.p) are specified by their absolute stereochemistry.

In a preferred embodiment the compound of formula (I) is a mixture of at least two enantiomers (I.a) to (I.p) or a mixture of the prodrugs or isotopically enriched forms thereof, or a mixture of the pharmaceutically acceptable salt or solvate thereof, wherein one enantiomer is enriched.

Preferably the compound of formula (I) is a mixture of (I.a) and (I.b) or a mixture of the pharmaceutically acceptable salts thereof, wherein the enantiomer excess (ee) of the enantiomer of formula (I.a) is at least 20%, preferably at least 50%, in particular at least 80%, especially at least 99%.

Preferred are compound of formula (I) according to the invention, or a prodrug or isotopically enriched forms thereof or a pharmaceutically acceptable salt thereof, wherein R¹ is CN.

Further, preferred are compound of formula (I) according to the invention, or a prodrug or isotopically enriched forms thereof or a pharmaceutically acceptable salt thereof, wherein R¹ is CN and R² is selected from hydrogen and F.

Further, preferred are compound of formula (I) according to the invention, or a prodrug or isotopically enriched forms thereof or a pharmaceutically acceptable salt thereof, wherein R² is hydrogen.

Further, preferred are compounds of formula (I) according to the invention, or a prodrug or a pharmaceutically acceptable salt or solvate thereof, wherein R² is F.

Another special embodiment are the compounds of formula (I), wherein R¹ and R² are selected from the definition given in one line of table 1:

TABLE 1

| Nr. | R¹ | R² |
|---|---|---|
| 1. | CN | F |
| 2. | CN | hydrogen |

Another special embodiment are the compounds selected from A, B and the mixture of each of the of compounds A to B with its respective enantiomer:

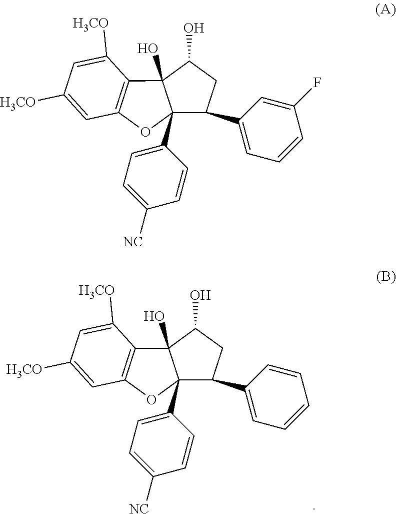 [SMILES: COc1cc(OC)c2c(c1)O[C@@]1(c3ccc(C#N)cc3)[C@H](c3cccc(F)c3)C[C@@H](O)[C@@]21O ; COc1cc(OC)c2c(c1)O[C@@]1(c3ccc(C#N)cc3)[C@H](c3ccccc3)C[C@@H](O)[C@@]21O]

Preferred is a compound of formula (A) or an enantiomeric mixture comprising the compounds of formula (A) and its enantiomer, in particular wherein the enantiomer excess (ee) of the enantiomer of formula (A) is at least 20%, preferably at least 50%, in particular at least 80%, especially at least 99%.

Preferred is a compound of formula (B) or an enantiomeric mixture comprising the compounds of formula (B) and its enantiomer, in particular wherein the enantiomer excess (ee) of the enantiomer of formula (B) is at least 20%, preferably at least 50%, in particular at least 80%, especially at least 99%.

Especially preferred is compound A.

Especially preferred is compound B.

The compounds of the present invention can be synthesized using the methods known in the prior art and together with methods known from synthetic organic chemistry, or variations thereof as appreciated by those skilled in the art.

### Virus

The present invention provides a compound of formulae (I), (I.a) or (I.b) or an enantiomeric mixture as defined herein and pharmaceutically acceptable salts thereof (“compounds of the invention”) for use in treatment or prevention of viral infections or diseases associated with viral infections.

The present invention provides a compound of formulae (I), (I.a) or (I.b) or an enantiomeric mixture as defined herein and pharmaceutically acceptable salts thereof (“compounds of the invention”) for use as an inhibitor of viruses.

Preferably, the disease or infection is selected from viral infections caused by positive single-stranded RNA viruses (+ssRNA viruses) and a negative single-stranded RNA viruses (−ssRNA viruses).

Positive-strand RNA viruses (+ssRNA viruses) are a group of related viruses that have positive-sense, single-stranded genomes made of ribonucleic acid. The positive-sense genome can act as messenger RNA (mRNA) and can be directly translated into viral proteins by the host cell's ribosomes. Positive-strand RNA viruses encode an RNA-dependent RNA polymerase (RdRp) which is used during replication of the genome to synthesize a negative-sense antigenome that is then used as a template to create a new positive-sense viral genome.

Especially, the (+) ssRNA virus is a member of a family selected from togaviridae, flaviviridae, coronaviridae and retroviridae.

Togaviridae is a family of enveloped positive-strand RNA viruses. It currently includes two genuses of enveloped viruses with a single-stranded RNA with positive polarity. The togaviridae include virus species in the genus alphavirus and rubella virus. Alphavirus transmits diseases to humans and animals through insects. Rubella virus transmits only between humans via the respiratory route.

Flaviviridae is a family of enveloped positive-strand RNA viruses which mainly infect mammals and birds. They are primarily spread through arthropod vectors (mainly ticks and mosquitoes). The flaviviridae include virus species in the genus hepaciviruses and flaviviruses. Diseases associated with this group include hepatitis, Dengue fever, Japanese encephalitis, Kyasanur Forest disease, Powassan virus, West Nile fever, Yellow fever and Zika fever.

Coronavirus is the common name for Coronaviridae and Orthocoronavirinae, also called Coronavirinae. Coronaviridae is a family of enveloped, positive-strand RNA viruses. Coronaviruses cause diseases in mammals and birds. In humans, the viruses cause respiratory infections.

The coronaviridae include virus species in the genus alphacoronavirus, betacoronavirus and torovirus. Preferably, the coronaviridae is selected from SARS-CoV, SARS-CoV-2, MERS-CoV, HCoV-229E, HCoV-NL63, HCoVC43 and HKU1.

Four human coronaviruses cause typically minor symptoms of a common cold, while three are known to cause more serious illness and can be lethal: SARS-CoV-1, which causes SARS; MERS-CoV, which causes MERS; and SARS-CoV-2, which causes COVID-19.

Severe acute respiratory syndrome coronavirus 1 (SARS-CoV-1 or SARS-CoV) is a strain of coronavirus that causes severe acute respiratory syndrome (SARS). It causes an often severe illness and is marked initially by systemic symptoms of muscle pain, headache, and fever, followed in 2-14 days by the onset of respiratory symptoms, mainly cough, dyspnea, and pneumonia. Another common finding in SARS patients is a decrease in the number of lymphocytes circulating in the blood. SARS-CoV is a member of the genus betacoronavirus and subgenus embecovirus.

Severe acute respiratory syndrome coronavirus 2 (SARS-CoV-2) is a strain of coronavirus that causes COVID-19, a respiratory illness. SARS-CoV-2 is a virus of the species severe acute respiratory syndrome-related coronavirus (SARS-CoV), related to the SARS-CoV-1 virus. SARS-CoV-2 is a member of the genus betacoronavirus and subgenus embecovirus.

Middle East respiratory syndrome-related coronavirus (MERS-CoV) is the virus that causes Middle East respiratory syndrome (MERS). It is a species of coronavirus which infects humans, bats, and camels. The species is a member of the genus Betacoronavirus and subgenus Merbecovirus.

Human coronavirus 229E (HCoV-229E) is a species of coronavirus which infects humans and bats. It is one of the viruses responsible for the common cold. HCoV-229E is a member of the genus alphacoronavirus and subgenus duvinacovirus.

Human coronavirus NL63 (HCoV-NL63) is a species of coronavirus, specifically a Setracovirus from among the Alphacoronavirus genus. Infection with the virus has been confirmed worldwide, and has an association with many common symptoms and diseases. Associated diseases include mild to moderate upper respiratory tract infections, severe lower respiratory tract infections, croup and bronchiolitis.

Human coronavirus OC43 (HCoV-OC43) is a member of the species Betacoronavirus 1, which infects humans and cattle. OC43 is one of seven coronaviruses known to infect humans. It is one of the viruses responsible for the common cold. It is of the genus betacoronavirus and subgenus embecovirus.

Human coronavirus HKU1 (HCoV-HKU1) is a species of coronavirus in humans and animals. It causes an upper respiratory disease with symptoms of the common cold, but can advance to pneumonia and bronchiolitis. HCoV-HKU1 is a member of the genus betacoronavirus and subgenus embecovirus.

Retroviridae is a family of enveloped positive-strand RNA viruses. The retroviridae include virus species in the genus inter alia deltretrovirus and lentivirus. Lentivirus causes chronic and deadly diseases characterized by long incubation periods, in humans and other mammalian species. The genus includes the human immunodeficiency virus (HIV), which causes AIDS.

Negative-strand RNA viruses (−ssRNA viruses) are a group of related viruses that have negative-sense, single-stranded genomes made of ribonucleic acid. They have genomes that act as complementary strands from which messenger RNA (mRNA) is synthesized by the viral enzyme RNA-dependent RNA polymerase (RdRp). During replication of the viral genome, RdRp synthesizes a positive-sense antigenome that it uses as a template to create genomic negative-sense RNA. Negative-strand RNA viruses also share a number of other characteristics: most contain a viral envelope that surrounds the capsid, which encases the viral genome, −ssRNA virus genomes are usually linear, and it is common for their genome to be segmented.

Preferably, the (−) ssRNA virus is a member of a family selected from arenaviridae, bornaviridae, bunyviridae, filoviridae, othymyxoviridae, paramyxoviridae and pneumoviridaerhabdoviridae, in particular filoviridae.

Preferably, filoviridae is selected from Bundibugyo ebolavirus, Reston ebolavirus, Sudan ebolavirus, Taï Forest ebolavirus, Zaire ebolavirus and Bombali ebolavirus.

As defined above positive single-stranded RNA viruses ((+) ssRNA) and negative single-stranded RNA viruses ((−) ssRNA) account for a large fraction of known viruses, including many pathogens as well as less clinically serious pathogens such as the rhinoviruses that cause the common cold. Therefore, in one embodiment the infection or disease is a infection or disease caused by positive single-stranded RNA viruses ((+) ssRNA) and negative single-stranded RNA viruses ((−) ssRNA).

In a preferred embodiment, infections or diseases are selected from dengue fever, MERS, COVID-19, SARS, ebola fever, AIDS and zika fever.

Diseases associated with positive-sense single-stranded RNA viral infections and negative-sense single-stranded RNA viral infections include various complications arising therefrom. Complications include respiratory distress, pulmonary fibrosis, pneumonia, cytokine storm, acute liver injury, septic shock, acute kidney injury, pancreatic injury, peripheral nervous systems complications (such as an impaired ability to taste, to smell, and vision impairment), muscle pain, inflammation of cardiac muscle, blood clots in veins, decreased blood flow in coronary arteries, cardiogenic shock, heart failure, impaired consciousness, brain inflammation, irritation and swelling of brain and blood vessels, acute cerebrovascular complications (such as stroke, seizures and slurred speech), arrhythmia, myocarditis, thrombotic events, rhabdomyolysis, neurocognitive deficits, and sensory and motor deficits. The present invention embraces the complications caused by SARS-CoV-2 infection.

In one embodiment the disease is lung inflammation. Suitably, the lung inflammation is caused by pathogenic infection, bacterial infection, fungal infection or viral infection, in particular a (+) ssRNA virus infection. More suitably, the lung inflammation is caused by a disease selected from the group consisting of pneumonia, acute respiratory disease symptom (ARDS), COPD, asthma, idiopathic pulmonary fibrosis, allergic rhinitis, rhinitis and sinusitis. More suitably, the lung inflammation is caused by CORD, asthma or idiopathic pulmonary fibrosis. Even more suitably, the lung inflammation is caused by COPD. Even more suitably, the lung inflammation is caused by asthma. Even more suitably, the lung inflammation is caused by idiopathic pulmonary fibrosis.

In one embodiment, the compounds of the invention are used for the treatment of or prevention of hyperinflammation associated with positive-sense single-stranded RNA viral infections, in particular as coronavirus infection. Preferably, the compounds according to the invention reduce hyperinflammation associated with coronavirus infection.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The phrase “therapeutically effective” is intended to qualify the amount of each agent, which will achieve the goal of improvement in disorder severity and the frequency of incidence, while avoiding adverse side-effects typically associated with alternative therapies. For example, effective antiviral agents prolong the survivability of the patient or his/her life quality, inhibit the rapidly multiplication of the viruses.

The terms “treat,” “treating,” and “treatment,” as used herein, refer to any type of intervention or process performed on, or administering an active agent to, the subject with the objective of reversing, alleviating, ameliorating, inhibiting or slowing down or preventing the progression, development, severity or recurrence of a symptom, complication, condition or biochemical indicia associated with a disease. By contrast, “prophylaxis” or “prevention” refers to administration to a subject who does not have a disease to prevent the disease from occurring.

As used herein, the term “cell” is meant to refer to a cell that is in vitro, ex vivo or in vivo. In the sense of the invention, an ex vivo cell can be part of a tissue sample excised from an organism such as a mammal. In the sense of the invention, an in vitro cell can be a cell in a cell culture. In the sense of the invention, an in vivo cell is a cell living in an organism such as a mammal.

The term “patient” includes humans and animals that receive either therapeutic or prophylactic treatment.

The term “subject” includes any human or animal. For example, the methods and compositions herein disclosed can be used to treat a subject having cancer.

A (non-human) animal includes all vertebrates, e.g. mammals and non-mammals, including cows, sheep, pigs, goats, horses, poultry, dogs, cats, non-human primates, rodents etc.

In one embodiment, the subject is a human subject.

The phrase “pharmaceutically acceptable carrier” as used herein means a pharmaceutically acceptable material, composition or vehicle, such as a liquid or solid diluent, solvent, excipient, manufacturing aid (e.g. lubricant) or encapsulating material, involved in carrying or transporting the subject compound from one organ, or portion of the body, to another organ, or portion of the body. Each carrier must be “acceptable” in the sense of being compatible with the other ingredients of the formulation.

Suitable other ingredients are the aforementioned carrier and further additives, including adjuvants, preserving agents, fillers, flow regulating agents, disintegrating agents, wetting agents, emulsifying agents, suspending agents, sweetening agents, flavoring agents, bittering agents, perfuming agents, antibacterial agents, antifungal agents, lubricating agents, dispensing agents, etc. Suitable additives are selected depending on the nature of the mode of administration and dosage forms; and not injurious to the patient.

The term “pharmaceutical composition” means a composition comprising a compound according to the invention in combination with at least one further compound selected from
a) at least one further pharmaceutically active substance and
b) at least one additional pharmaceutically acceptable carrier and or additive.

For use in therapy at least one compound of formulae (I), (I.a) or (I.b) or an enantiomeric mixture as defined above or a pharmaceutically acceptable salt or solvate thereof or a pharmaceutically acceptable carrier are usually administered as a pharmaceutical composition. The invention also relates to a pharmaceutical composition comprising at least one compound of formulae (I), (I.a) or (I.b) or an enantiomeric mixture as defined above or a pharmaceutically acceptable salt or solvate thereof and a pharmaceutically acceptable carrier.

The compounds according to the invention may be administered by any convenient method, e.g. by oral, parenteral, buccal, sublingual, nasal, rectal, intrathecal or transdermal administration or by inhalation (e.g. for topical administration to the lung by inhalation), and the pharmaceutical compositions adapted accordingly.

A compound according to the invention, which is active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, a preservative, a flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be optimized and then reconstituted with a suitable solvent just prior to administration.

Compositions for nasal administration or for inhalation (e.g. for topical administration to the lung by inhalation) may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multi-dose quantities in sterile form in a sealed container which can take the form of a cartridge or refill for use with an atomising device.

Alternatively the sealed container may be a disposable dispensing device such as a single dose nasal inhaler or an aerosol dispenser fitted with a metering valve suited for delivery of the aerosol to the nasal or bronchial passages. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Topical administration to the lung may also be achieved by use of a dry-powder formulation which contains the compound of the invention in finely divided form optionally together with one or more carriers or other excipients. A dry powder formulation is typically delivered using a dry powder inhaler (DPI) device.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches. In one embodiment the composition is in unit dose form such as a tablet, capsule or ampoule.

In one embodiment of the invention, the compound of formulae (I), (I.a) or (I.b) or an enantiomeric mixture as defined above is used in combination with a further therapeutic agent or agents. When at least one compound of formulae (I), (I.a) or (I.b) or an enantiomeric mixture as defined above are used in combination with other therapeutic agents, the compound of formulae (I), (I.a) or (I.b) or an enantiomeric mixture as defined above may be administered either sequentially or simultaneously by any convenient route. Alternatively, the compounds may be administered separately.

The invention will be illustrated further with reference to the examples that follow, without restricting the scope to the specific embodiments described. The invention includes all combinations of described and especially of preferred features that do not exclude each other.

### DESCRIPTION OF THE DRAWINGS

FIG. 1: The mRNA of Dual reporter assay is shown. Firefly luciferase (F Luc) will be translated by eIF4F complex and Renilla Luciferase (R Luc) will be translated independently of the complex.

FIG. 2: The sequence of 5′UTR of mRNA of virus are shown.

FIG. 3: Dual luciferase assay for Cap-dependent translation initiation

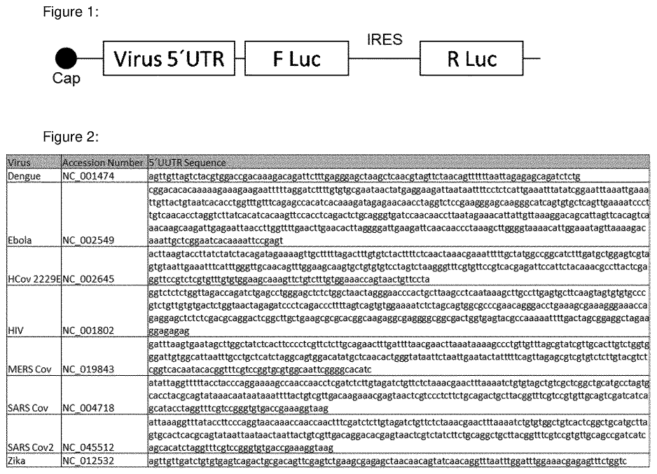

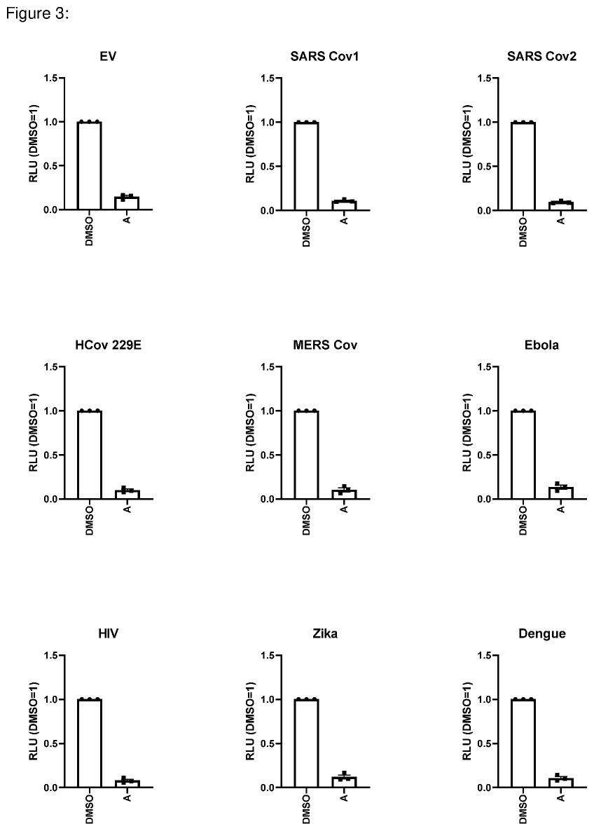

A Plasmid MAP of the dual luciferase assay system. B Dual luciferase assay for Cap-dependent translation initiation was performed in HeLa cells transfected with dual luciferase reporter gene based on pFR_HCV_xb. Cells were treated with compound A (200 nM) for 24 h in serum-free DMEM. After incubation, Dual luciferase reporter assay was performed according to the manufacturer's instruction and the luminescence was measured using a multiplate reader. Data were normalized to cells transfected with the indicated mutant and exposed to DMSO for 24 h. DMSO-treated cells were set as 1. The bars represent mean±SEM from 3 independent experiments. (EV corresponds to empty vector)

### Dual Luciferase Assay

Hela Cells were cultured in DMEM (10% heat inactivated FBS, 1 mM Sodium Pyruvate) and seeded at 1×10⁶ cells/ml, 2 ml in 6 well cell culture plate in growth medium. 2 μg of plasmids were transfected into cells with 0.5 mM of PEI reagent in 200 μl PBS. One day after transfection, cells were harvested and seeded into 96 well white plates, half area (Greiner). After an additional day the medium was changed to serum free DMEM and the cells were incubated for 24 h with Compounds. Dual-Glo Luciferase Assay was performed according to the manufacturer's instructions (Promega, N2920). The luminescence was measured using Tecan infinite (Tecan).

### EXAMPLES

The compound A relates to

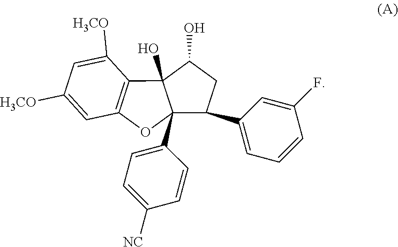 [SMILES: COc1cc(OC)c2c(c1)O[C@@]1(c3ccc(C#N)cc3)[C@H](c3cccc(F)c3)C[C@@H](O)[C@@]21O]

1. A compound of formula (I) 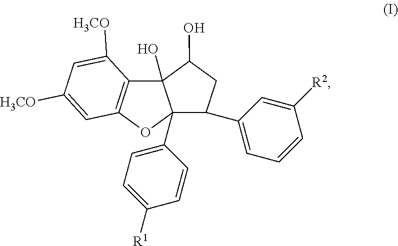
prodrugs or isotopically enriched forms thereof or a pharmaceutically acceptable salt or solvate thereof, wherein
R¹ is selected from CN and hydrogen,
R² is selected from hydrogen and halogen,
for use in treatment or prevention of viral infections or diseases associated with viral infections infection and/or for use as inhibitor of viruses, wherein the infection or disease is selected from viral infection caused by positive single-stranded RNA viruses ((+) ssRNA) and a negative single-stranded RNA viruses ((−) ssRNA).

2. A compound of formula (I) 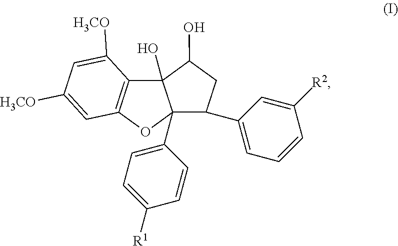
prodrugs or isotopically enriched forms thereof or a pharmaceutically acceptable salt or solvate thereof, wherein
R¹ is selected from CN and hydrogen,
R² is selected from hydrogen and halogen,
for use in treatment or prevention of viral infections or diseases associated with viral infections.

3. The compound of formula (I) according to claim 1 or 2, for use as an inhibitor of viruses.

4. The compounds of formula (I) for use in treatment or prevention of viral infections or diseases associated with viral infections and/or for use as an inhibitor of viruses according to claims 1 to 3 selected from compounds of formula (I.a) or (I.b) or an enantiomeric mixture comprising the compounds of formula (I.a) and (I.b) or prodrugs or isotopically enriched forms thereof or the pharmaceutically acceptable salts or solvates thereof, 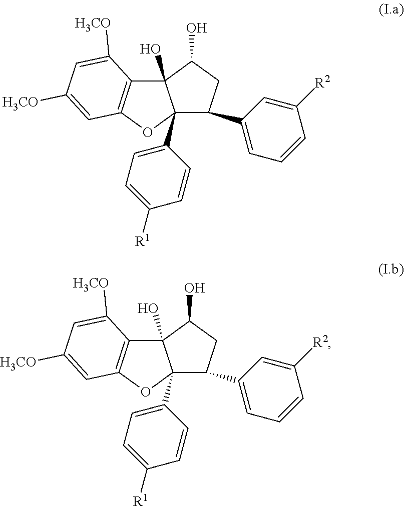
wherein R¹ and R² have the same meanings as defined in claim 1.

5. The compounds of formula (I) for use in treatment or prevention of viral infections or diseases associated with viral infections and/or for use as an inhibitor of viruses according to any of the preceding claims, being a mixture of (I.a) and (I.b), prodrugs or isotopically enriched forms thereof or a pharmaceutically acceptable salts or solvate thereof, wherein the enantiomeric excess (ee) of the enantiomer of formula (I.a) is at least 20%, preferably at least 50%, in particular at least 80%, especially at least 99%.

6. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as an inhibitor of viruses, according to any of the preceding claims, wherein R¹ is CN.

7. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as an inhibitor of viruses, according to any of the preceding claims, wherein R² is selected from hydrogen and F.

8. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as inhibitor of viruses, according to any of the preceding claims, selected from compounds of the formulae A and B and the mixture of each of compound A to B with its respective enantiomer,

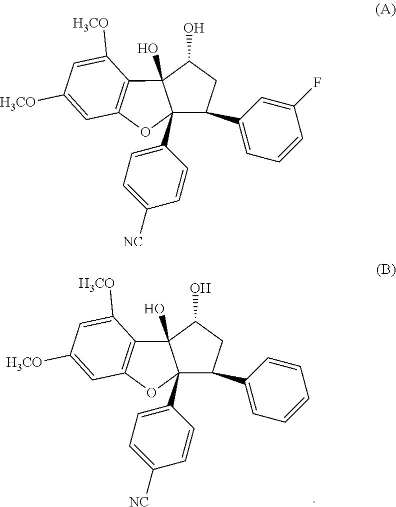 [SMILES: COc1cc(OC)c2c(c1)O[C@@]1(c3ccc(C#N)cc3)[C@H](c3cccc(F)c3)C[C@@H](O)[C@@]21O ; COc1cc(OC)c2c(c1)O[C@@]1(c3ccc(C#N)cc3)[C@H](c3ccccc3)C[C@@H](O)[C@@]21O]

9. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as inhibitor of viruses, according to any of the preceding claims, is selected from compound of A and the mixture of compound A with its respective enantiomer,

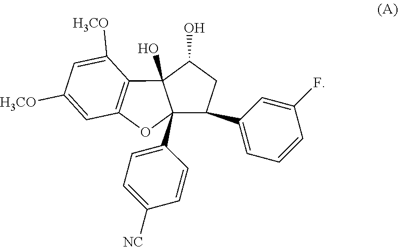 [SMILES: COc1cc(OC)c2c(c1)O[C@@]1(c3ccc(C#N)cc3)[C@H](c3cccc(F)c3)C[C@@H](O)[C@@]21O]

10. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as an inhibitor of viruses, according to any of the preceding claims wherein the (+) ssRNA virus is a member of a family selected from togaviridae, flaviviridae, coronaviridae and retroviridae.

11. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as an inhibitor of viruses, according to claims 1 to 9, wherein the (−) ssRNA virus is a member of a family selected from arenaviridae, bornaviridae, bunyviridae, filoviridae, othymyxoviridae, paramyxoviridae pneumoviridae and rhabdoviridae, in particular filoviridae.

12. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as an inhibitor of viruses, according to any of claims 1 to 10, wherein flaviviridae is selected from Dengue virus, Japanese encephalitis, Kyasanur Forest disease, Powassan virus, West Nile virus, Yellow fever virus, and Zika virus.

13. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as an inhibitor of viruses, according to claims 1 to 10, wherein coronaviridae is selected from SARS-CoV, SARS-CoV-2, MERS-CoV, HCoV-229E, HCoV-NL63, HCoVC43 and HKU1.

14. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as an inhibitor of viruses, according to claims 1 to 10, wherein retroviridae is selected from HIV-1 and HIV-2.

15. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as an inhibitor of viruses, according to claims 1 to 9 and 11, wherein filoviridae is selected from Bundibugyo ebolavirus, Reston ebolavirus, Sudan ebolavirus, Taï Forest ebolavirus, Zaire ebolavirus and Bombali ebolavirus.

16. The compounds of formulae (I) or (I.a) or (I.b) or an enantiomeric mixture comprising the compounds of formula (I.a) and (I.b) or prodrugs or isotopically enriched forms thereof or a pharmaceutically acceptable salt or solvate thereof, for use in treatment or prevention of viral infections or diseases associated with viral infections and/or for use as an inhibitor of viruses, according to any of claims 1 to 10 and 12 to 14, wherein the disease or infection is selected from dengue fever, MERS, COVID-19, SARS, ebola fever, AIDS and zika fever.
